(19) United States
(12) Patent Application Publication
(10) Pub. No.: US 2011/0230679 A1
(43) Pub. Date: Sep. 22, 2011

(54) Reactive Static Mixer

(75) Inventors: Paul A. Gillis, Lake Jackson, TX (US); Joerg-Peter Gehrke, Stade (DE); Artur Klinger, Stade (DE)

(73) Assignee: DOW GLOBAL TECHNOLOGIES, INC., Midland, MI (US)

(21) Appl. No.: 12/725,266

(22) Filed: Mar. 16, 2010

(51) Int. Cl. C07C 211/00; B01J 19/00

(52) U.S. Cl. 564/305; 422/224

(57) ABSTRACT

This disclosure relates to a static phosgene mixer, and more generally, to an apparatus for mixing of fluid components such as phosgene and amine during an highly reactive, chemical reaction that is vulnerable to the creation of undesired by-products, and equipment fouling. A guide element is disposed in the static mixer to divert the incoming flow of phosgene around the guide element and create an annular mixing passage in the static mixer. This allows for the use of an increased external radius of the effective phosgene flow while maintaining phosgene velocity by creating a blockage of the flow. The same flow, when transformed from a circular configuration to an annular configuration has an increased external radius, and a greater quantity of MDA jets can be placed along the increased radius, thus increasing the overall homogeneity of the mixture. Further, the cross-sectional area of the annular passage section of phosgene defined around the guide element controls the velocity of phosgene which facilitates the mixing of MDA injected through the jets into the phosgene.

### FIELD OF THE DISCLOSURE

This disclosure relates to a static mixer, and more generally, to an apparatus for mixing of fluid components such as phosgene and amine during a highly reactive, chemical reaction producing undesirable by-products and equipment fouling.

### BACKGROUND

The field of conventional mixing devices can be roughly divided into two main areas: dynamic or mechanical mixers and static mixers. Dynamic or mechanical mixers rely on some type of moving part or parts to ensure the desired or thorough mixing of the reactants. Static mixers generally have no prominent moving parts and instead rely on pressure differentials within the fluids being mixed to facilitate mixing. The current disclosure is directed to a static mixer.

The inventor of the current disclosure is also the inventor of U.S. patent application Ser. No. 11/658,193 directed to a tapered aperture multi-tee mixer. In this application, multi-tee mixers include a tee-pipe junction and a straight pipe section with nozzles and blind flanges for the rapid initiation of the chemical reaction. The junction at these prior art multi-tee static mixers includes a mixing chamber having separate inlets for at least two components and an outlet. The inlet for one of the components is defined along a longitudinal axis of the multi-tee mixer and the inlet for the other component(s) is formed as a plurality of nozzles or jets disposed around the circumference of the mixing chamber and oriented normal to the longitudinal axis of the multi-tee mixer.

The quality of the products prepared in a prior art apparatus depends on the quality and rate of mixing of the fluid components. For example, in the case of phosgene chemistry, Methylenedi(phenylamine) (MDA) is mixed with COCl₂ (Phosgene) to create a mixture of Hydrochloric Acid (HCl) and Carbamyl Chlorides, and the carbamyl chlorides decomposing to methylnediphenyl dissocyanate (MDI) and HCL. While the production of HCI and Carbamyl Chlorides is desired, secondary reactions can lead to the creation of undesired by-products such as urea. Since the formation of urea is undesirable, the increase of the ratio of phosgene to MDA, a dilution of MDA, or a proper mixing minimizes the formation of undesired by-products such as urea.

The quality and rate of mixing can be affected by fouling, caking, or plugging of the jets of the inlet of the mixer tee and results in decreased performance. Over the course of time, caking and subsequent clogging disturbs the injection and the distribution of flow through the inlet jets for MDA in static mixers.

Caking may also occur on the side surfaces of jets as a result of secondary reactions. When caking and/or clogging occur, a continuous process has to be interrupted and the static mixers taken apart and cleaned. This results in undesirable idle periods. Where hazardous substances are used, industrial hygiene regulations necessitate expensive measures during the disassembly of the static mixers, such as the thorough flushing of the system before disassembly, exhaustion of the atmosphere, protective clothing, and breathing apparatuses for the workers. Each of these measures adds to the overall cost, reduces throughput, and reduces the efficiency of the process.

Some chemical reactions require proper mixing to reduce secondary reactions. Improper mixing can allow a product of an initial reaction to react with another component in the reaction stream to generate an undesired product, as illustrated in one example above. Improper mixing may also contribute to equipment fouling. Consequently, mixer designs that do not account for proper mixing can result in lower overall yield of the desired product or can generate a product that clogs or fouls the reactor system leading to down time and/or increased maintenance costs.

In a mixer from the prior art as shown in FIG. 1A, the phosgene is transported along the longitudinal axis of the device and the MDA is inserted from the top orifice into the main stream of phosgene. Another means of mixing is shown at FIG. 1B which teaches the use of tapered amine jets to avoid phosgene stream concentrations and expansions. While this static mixer is an improvement over the prior art, further improvements may be made. For example, the design can be improved to better accommodate changes in the flow rates of the two reactant streams. In the prior configurations, higher amine flows could result in the streams from the opposite amine jets flowing into each other. As the velocity of phosgene steam is increased, the depth of amine jet penetration is reduced. Furthermore, increased stream flows change stream pressure drops and the pressure drop of one stream requires a pressure change of the other stream to maintain reaction stoichiometry. In order to overcome the disadvantages of the prior art, what is needed is a static mixer with an internal configuration that allows for an increased passage of phosgene while controlling precisely the mixing of MDA in the phosgene.

### SUMMARY

This disclosure relates to a static mixer, and more generally, to an apparatus for mixing of fluid components such as phosgene and amine during an highly reactive, chemical reaction that is vulnerable to the creation of undesired by-products, and equipment fouling. A guide element is disposed in the static mixer to divert the incoming flow of phosgene around the guide element and create an annular mixing passage in the static mixer. This allows for the use of an increased external radius of the effective phosgene flow while maintaining phosgene velocity by creating a blockage of the flow. The same flow, when transformed from a circular configuration to an annular configuration has an increased external radius, and a greater quantity of MDA jets can be placed along the increased radius, thus increasing the overall homogeneity of the mixture. Further, the cross-sectional area of the annular passage section of phosgene defined around the guide element controls the velocity of phosgene which facilitates the mixing of MDA injected through the jets into the phosgene.

### BRIEF DESCRIPTION OF THE DRAWINGS

Certain preferred embodiments are shown in the drawings. However, it is understood that the present disclosure is not limited to the arrangements and instrumentality shown in the attached drawings.

FIGS. 1A, and 1B are cross-sections of a static mixer from the prior art.

FIG. 2 is a cross-section of a static mixer with guide element according to an embodiment of the present disclosure.

FIG. 3 is an isometric view of a static mixer according to an embodiment of the present disclosure.

FIG. 4 is a side view with dashed internals of the static mixer of FIG. 3.

FIG. 5 is a flow diagram of the phosgene and MDA flows within a static mixer from the prior art according to an embodiment of the present disclosure.

FIG. 6 is a flow diagram of the phosgene and MDA flows within a static mixer such as shown at FIG. 2 according to another embodiment of the present disclosure.

FIG. 7 is a cross-section of a static mixer with a rectangular cavity according to another embodiment of the present disclosure.

### DETAILED DESCRIPTION

For the purposes of promoting and understanding the invention and principles disclosed herein, reference is now made to the preferred embodiments illustrated in the drawings, and specific language is used to describe the same. It is nevertheless understood that no limitation of the scope of the invention is hereby intended. Such alterations and further modifications in the illustrated devices and such further applications of the principles disclosed as illustrated herein are contemplated as would normally occur to one skilled in the art to which this disclosure relates.

Two embodiments are described in detail. The first embodiment is shown in FIGS. 2-6 and the second embodiment is shown in FIG. 7. One of ordinary skill in the art will recognize that there is an infinite number of alternative geometric variation and that this disclosure is not limited to any certain geometry described herein. In one embodiment, an internal guide element identified as reference numeral 5 in FIG. 2 is disposed in the center of the continuous phosgene flow as indicated by the arrows 20 in FIG. 2 to intensify the phosgene flow, that is, increase the velocity and turbulence, for optimum mixing. In another embodiment, the external shape is of such configuration that no internal guide element is needed. It will be recognized that static mixing having generally circular and rectangular configurations are shown and that any other shape, geometry, or configuration may be used where the internal guide 5, is designed to create flow of phosgene of a quantifiable thickness.

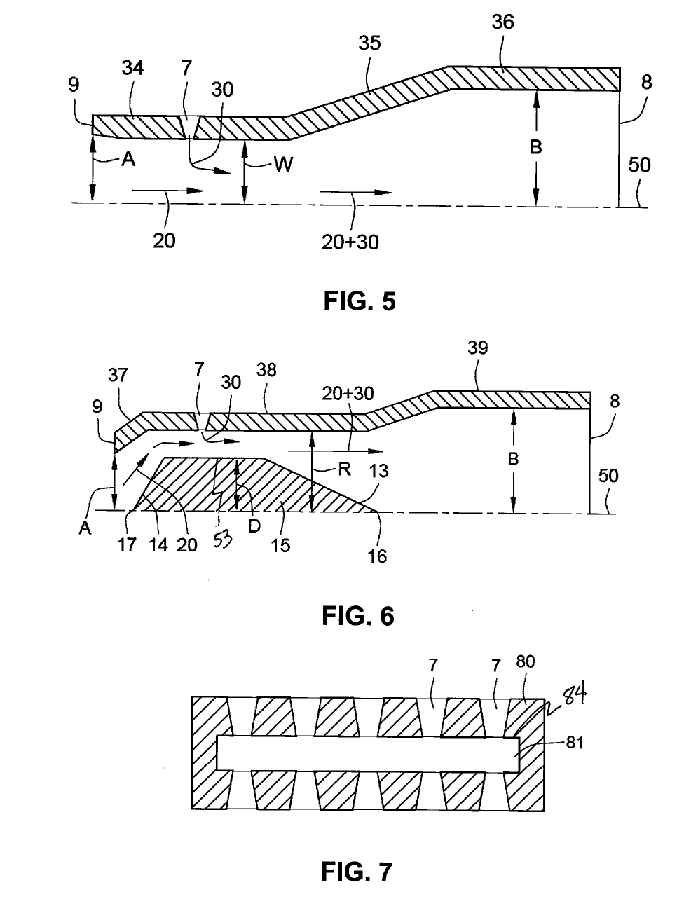

For example, the static mixer as shown in FIG. 7 could be connected to a circular inlet and a circular outlet. As a consequence of the change in the geometry of the flow of MDA 30 resulting from a circular guide element disposed in the continuous flow of phosgene as illustrated in FIG. 2, the flow of phosgene is concentrated from a circular flow of section S_{C}=πW², where W is the external radius of the phosgene flow or the inner surface 8 of a first passageway 9 as shown in FIG. 5, to an annular flow of section S_{S}=π(R²−D²), where R is an inner surface of the housing and D the external radius or outer surface of the guide element as shown in FIG. 6.

As shown in one embodiment in FIGS. 5 and 6, a flow of fluid such as MDA 30 may be released or injected through the second passageways 7 into the continuous flow of phosgene. The second passageways 7 may be formed to have a circular configuration or any other geometry, configuration, or shape as described fully in U.S. patent application Ser. No. 11/658,193, which is fully incorporated herein. In one preferred embodiment, R is greater than W and permits the circumferential distribution of a greater number of second passageways 7 around an annular flow geometry than around the initial circular flow geometry.

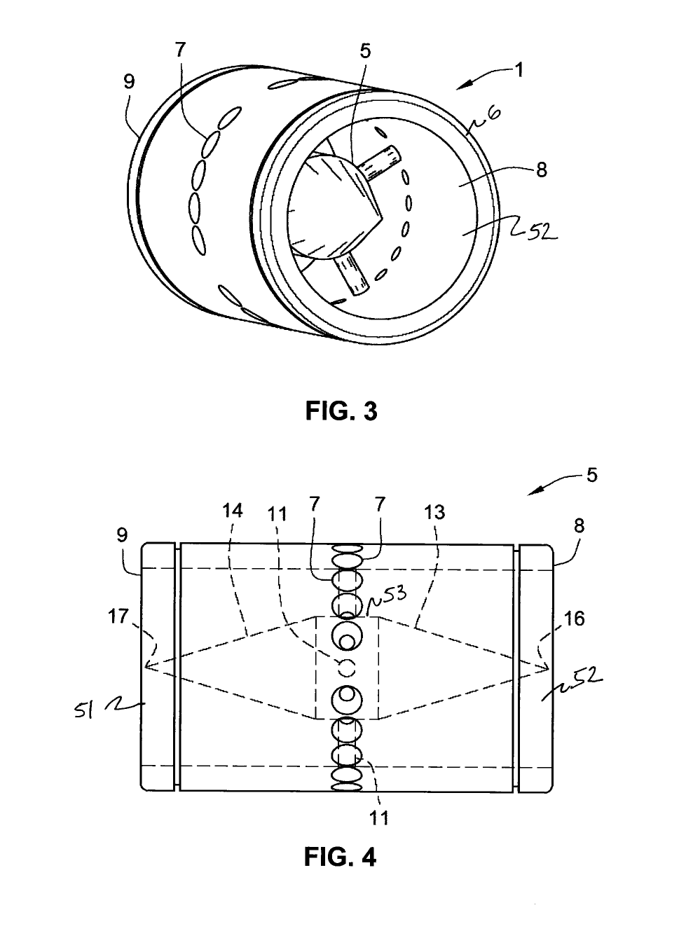

One of ordinary skill in the art will recognize a plurality of MDA jets may be placed about the circumference of the static mixer. In one embodiment as shown in FIG. 3, a quantity of twenty MDA jets is placed about the circumference or outer surface of the static mixer. One of ordinary skill in the art will recognize that only one embodiment is shown in FIGS. 2-5 and that use of any geometry, configuration, or shape may be used to obtain the optimal quantity and distribution of the second passageway 7. For example, FIG. 4 illustrates one embodiment where the rods 11 are disposed at the same radial location along the housing 2 as the second passageway 7. It is within the teachings of the present disclosure that an infinite number of various structures or mechanisms may be used to dispose the guide element in the housing 2. For example, two sets of rods 11 longitudinally offset from the second passageway 7, rods 11 having a flattened section, or any other structure or mechanism to dispose the guide element 5 in the housing 2 may be used.

The inlet opening A of the prior art, as shown in FIG. 5, may be defined to have a unitary radius of 0.875 and a cross-sectional area of Sw=0.76. The outlet opening B has the same radius as the radius of the inlet opening. The cross-sectional flow areas are S_{B}=4S_{A}. In an embodiment of the present invention, as shown in FIG. 6, S_{A} and S_{B} may be defined with a value of 1 and 2, respectively. However, S_{R-D}=S_{A}(R²−D²)=0.76S_{A} where D is 1.22 and R is 1.5, the value S_{R-D}=0.76S_{A} or S_{R-D}=S_{W}. In both configurations shown in FIGS. 5 and 6, respectively, the cross-sectional flow area near the second passageway 7 is the same. The surface area (L) where the second passages can be aligned along the circumference of the housing 2 increases from L=2πW to L=2πR or from 0.875 to 1.5 an increase of 70%. While one possible configuration is shown, any possible numerical variation from the described configuration is contemplated.

As shown in FIG. 6, a ratio defined by a radius of the annular straight section over a radius of the inner surface (D/R) is approximately 0.813. In another embodiment, the ratio is in the range of 0.25 to 0.95. In further embodiments, the ratio is from 0.6 to 0.9. The guide element 5 disposed in the flow of phosgene creates a pressure drop in the mixer 1 along both the first passageway 9, by forcing the flow of phosgene to flow around the guide element 5, as shown by the arrows on FIG. 3, and in the second passageway 7 by forcing the flow of MDA to travel sideways, as shown by the arrow 30 in FIG. 6. To reduce the pressure drop, the guide element 5 comprises a leading section 14, a trailing section 13, and an annular straight section 53 defined between the leading section 14 and the trailing section 13. In order to further reduce the pressure drop, both the leading section 14, and the trailing section 13 are preferably configured as cones, each having a tip 17, 16, respectively.

Simulations were done to determine the different pressure drops through the mixer on both the phosgene side (ΔP_{PHOS}) and the MDA side (ΔP_{AMINE}) and to determine the percentage of impurities by-products called Addition Product A (APA) for the tubular configuration of FIG. 5 and the annular configuration of FIG. 6 with different numbers of jets. The total cross-sectional area of the amine jets was held constant. The results are given in the following table:

| | APA (%) | ΔP_{PHOS} | ΔP_{AMINE} |
|---|---|---|---|
| Tubular: 1 Jet | 8.5 | 1X | 1Y |
| Annular: 1 Jet | 6.5 | 1.1X | 1.2Y |
| Annular: 2 Jets | 5.9 | 1.2X | 1.3Y |
| Annular: 3 Jets | 5.4 | 1.3X | 1.4Y |

As shown in the above table, a pressure baseline is calculated from the tubular configuration for 1 jet (1X and 1Y of pressure on both the MDA and the phosgene). For example, for the annular 2 jets configuration, the pressure drop on the phosgene line is 1.2× or 120% the baseline pressure, or an increase in 20% from the baseline. The 20% increase in pressure gradient also corresponds to an increase in pressure loss of the MDA of 30% from the baseline. The table above also shows an increase in pressure drop as more jets are used. Pressure losses may be undesirable and require greater power from the flow pump. Conversely, in the examples given above, the APA or the quantity of undesirable by-product decreases from 8.5% down to 5.4% as the annular configuration of jets changes. The table shows the capacity to determine an equilibrium point, based on system requirements, to optimize the acceptable quantity of APA based on acceptable pressure drop values.

On of ordinary skill in the art will recognize that only one possible configuration and geometry of housing 2 with guide element 5 is shown and that a large quantity of parameters have been changed to optimize the design based on the viscosity of the different fluids in the static mixer 1, the desired velocity/rate of production of a mixing compound, and the expansion coefficient of the compound being mixed.

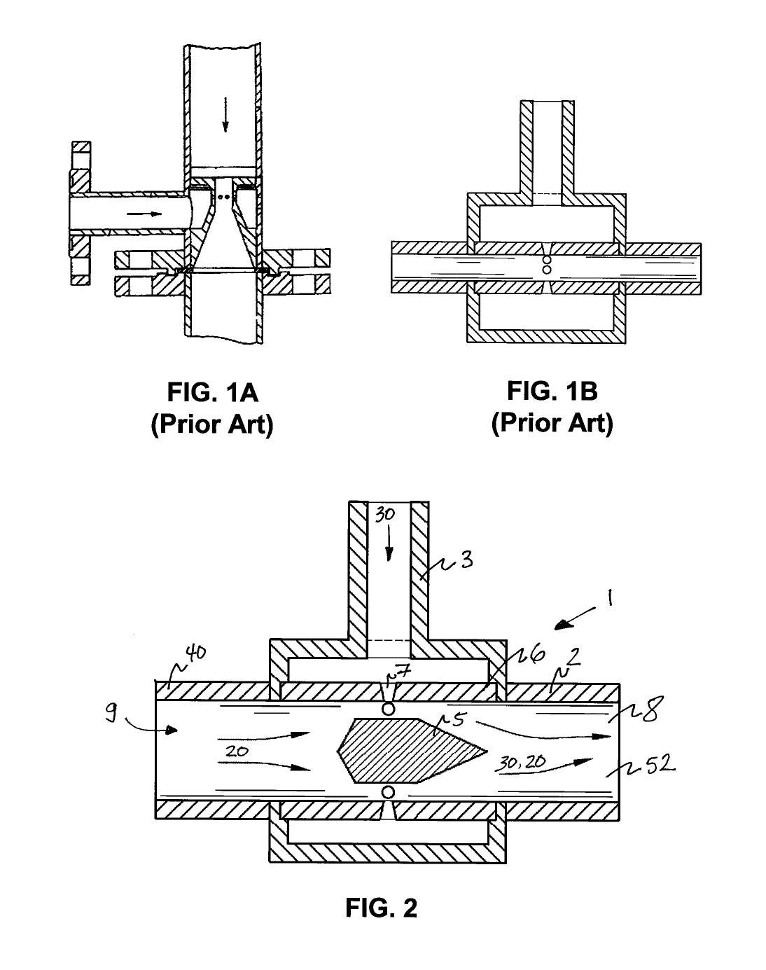

Obviously, different fluids will require different optimization values. The present disclosure is not limited to the elements or parameters disclosed herein. Additionally, it is within the teachings of the present disclosure that a prior art static mixer may be retrofitted with a static mixer of the present disclosure to improve performance by increasing the internal diameter and adding a guide element 5 to the static mixer 1. For example, the static mixer embodiment shown in FIG. 2 may be substituted for the prior are static mixer shown in FIG. 1. In the event of the internal radius of the first passageway of the prior art static mixer cannot be increased, the external diameter of the guide element 5 must be reduced in size and a configuration can be applied in accordance with the teachings of the present disclosure, to obtain the advantages described herein.

Returning to FIG. 2, the first passageway 9 is defined by an inner surface 8 formed in the housing 2, which extends along a longitudinal axis from right to left. The first passageway 9 including a first end 51 configured as an inlet and a second end 52 configured as an outlet to facilitate movement of a first fluid 20, such as phosgene, from the inlet to the outlet. The second passageway 7 is defined individually and collectively by a plurality of bores, as shown with greater specificity in FIG. 4. The bores are formed in the housing 2 in communication with the first passageway 9 and are disposed at a mixing location 53 between the first end 51 and the second end 52 to facilitate movement of a second fluid 30, such as MDA, from the second passageway 7 into the first passageway 9 to mix with the first fluid.

FIG. 4 shows a configuration where the second passageway 7, having twenty conical bores or a plurality of bores in this embodiment, is generally aligned with the annular straight section disposed on the outer surface 53 of the guide element 5. FIG. 4 illustrates an embodiment where the leading section 14 and the trailing section 13 are symmetrical. FIG. 7 illustrates another embodiment similar in function to those described herein but having a different configuration. The housing 80, and the inner surface 84 are generally rectangular. Such configuration may be dictated by an existing installation (i.e., retrofit), the fluids to be mixed, or other various reasons. One of ordinary skill in the art will recognize that the present disclosure is not limited to any specific geometry, configuration, or shape.

FIG. 2 shows is a static mixer 1 with a first passageway 9 defined by an inner surface 8 of a housing 2, a second passageway 7 defined by at least one bore that is in communication with the first passageway 9, and a guide element 5 disposed in the first passageway 9, generally aligned with the second passageway 7. An annular mixing chamber 67 is defined between the guide element 5 and the inner surface 8 adjacent the second passageway 7. The guide element 5 shown on FIG. 4 may also include a leading section 14, a trailing section 13, and an annular straight 55 section defined between the leading section 14 and the trailing section 13 as described above.

In yet another embodiment, a method of preventing improper-mixing within a rapid mixer and reducing the formation of by-products during phosgene and amine mixing is disclosed. Such method may comprise the steps of transporting a first fluid that may be a continuous phosgene stream 20 through a static mixer 1 including a housing 2 having a first passageway 9 and a second passageway 7. The first passageway 9 is defined by an inner surface 8 extending through the housing 2 along a longitudinal axis of the housing 2. A first end 51 of the first passageway 9 is configured as an inlet and a second end 52 is configured as an outlet in order to facilitate movement of a first fluid 20 from the inlet 51 to the outlet 52. The second passageway 7 is defined individually and collectively by a plurality of bores 7 formed in the housing 2 that is in communication with the first passageway 9 and one disposed at a mixing location 55 disposed between the first end 51 and the second end 52. A guide element 5 is disposed in the first passageway 9 and connected to the housing 2. The guide element 5 includes an outer surface 53 disposed adjacent the second passageway 7 to define an annular mixing chamber. Further, the method includes the steps of injecting a second fluid that may be a continuous stream amine MDA 30, as shown in FIG. 6, into the first passageway 9 through the plurality of bores 7 and mixing the first and second fluids, which may be phosgene and amine, 20+30 in FIG. 6 in the annular mixing chamber defined between the inner surface 8 and the guide element 5. In yet another embodiment, the continuous stream of amine at the step of injecting a continuous stream amine into the static mixer through the plurality of bores includes a portion of solvent and where the portion may be greater than the proportion of amine, such as for example up to 90% of the stream.

Persons of ordinary skill in the art appreciate that although the teachings of this disclosure have been illustrated in connection with certain embodiments and methods, there is no intent to limit the invention to such embodiments and methods. On the contrary, the intention of this disclosure is to cover all modifications and embodiments falling fairly within the scope the teachings of the disclosure.

1. A static mixer for liquids having a plug flow characteristic, comprising:
a housing including a first passageway with a first fluid and a second passageway with a second fluid;
the first passageway defined by an inner surface through the housing extending along a longitudinal axis of the housing, the first passageway including a first end configured as an inlet and a second end configured as an outlet to facilitate movement of the first liquid from the inlet to the outlet,
the second passageway defined individually and collectively by a plurality of bores formed in the housing, the bores being in communication with the first passageway disposed at a mixing location between the first end and the second end to facilitate movement of the second liquid from the second passageway into the first passageway to radially mix with the first liquid with the plug flow characteristic without swirl or back mixing along the longitudinal axis in the direction of the inlet, and
a guide element disposed in the first passageway and connected to the housing, the guide element including an outer surface disposed adjacent the second passageway to define a perimeter mixing chamber between the inner surface and the outer surface.

2. The static mixer of claim 1, wherein the guide element comprises a leading section, a trailing section, and an annular straight section defined between the leading section and the trailing section.

3. The static mixer of claim 1, wherein the housing and the inner surface are generally circular.

4. The static mixer of claim 1, wherein the second passageway comprises a plurality of bores distributed around the housing or barrel.

5. The static mixer of claim 2, wherein the second passageway is generally aligned with the annular straight section.

6. The static mixer of claim 2, wherein a ratio defined by a radius of the annular straight section over a radius of the inner surface is in the range of greater than 0.30 to approximately 0.95.

7. The static mixer of claim 2, wherein a ratio defined by a radius of the annular straight section over a radius of the inner surface is approximately 0.8.

8. The static mixer of claim 2, wherein the leading section and the trailing section are each configured as right circular cones.

9. The static mixer of claim 1, wherein the housing, the inner surface, and the guide element are generally rectangular.

10. The static mixer of claim 1, wherein the first fluid is phosgene, and the second fluid include at least a portion of an amine.

11-20. (canceled)
